(19) United States
(12) Patent Application Publication
(10) Pub. No.: US 2007/0254374 A1
(43) Pub. Date: Nov. 1, 2007

(54) WATER QUALITY ANALYZER

(75) Inventors: Takeshi Iharada, Kyoto-shi (JP); Yozo Morita, Kyoto-shi (JP)

Correspondence Address: SUGHRUE MION, PLLC, 2100 PENNSYLVANIA AVENUE, N.W., SUITE 800, WASHINGTON, DC, 20037, US

(73) Assignee: SHIMADZU CORPORATION, Kyoto-shi (JP)

(21) Appl. No.: 11/776,218

(22) Filed: Jul. 11, 2007

(30) Foreign Application Priority Data
Sep. 26, 2005 (JP) 2005-278818

(51) Int. Cl. G01N 33/00

(52) U.S. Cl. 436/146

(57) ABSTRACT

Disclosed is a water quality analyzer, which comprises a total organic carbon (TOC) measurement section for converting a carbon component in an aqueous sample to carbon dioxide and measuring an amount of the carbon dioxide, a conductivity measurement section for measuring a conductivity of an aqueous sample, a gas aeration mechanism for passing a degassing gas devoid of carbon dioxide gas through an aqueous sample to perform a degassing treatment of expelling carbon dioxide gas in the aqueous sample, and a control section for controlling respective operations of TOC measurement and conductivity measurement. The control section is operable, just before measuring a conductivity of a specific aqueous sample during the conductivity measurement for the specific aqueous sample, to controllably instruct the gas aeration mechanism to pass the degassing gas through the specific aqueous sample so as to perform the degassing treatment.

### BACKGROUND OF THE INVENTION

1. Field of the Invention

The present invention relates to an analyzer for managing target water based on conductivity. Conductivity reflects a total ion content in water and serves as a critical evaluation item of water quality, and a typical sampling target is pure water (including ultra-pure water). Pure water as a measurement target includes cleaning waters for use in semiconductor or liquid crystal manufacturing processes, pharmaceutical waters such as water for injection, and cooling waters for use in nuclear electric plants.

2. Description of the Related Art

In an operation of measuring a conductivity of a carbon dioxide gas-soluble aqueous sample, such as pure water, the aqueous sample is likely to absorb carbon dioxide gas in ambient air and create bicarbonate ions therein. Thus, the conductivity measurement has to be completed before the aqueous sample comes into contact with ambient air to cause mixing of carbon dioxide gas. For example, in an operation of measuring a conductivity of pure water supplied through a water-supply line, the pure water in the line can avoid contact with ambient air. Therefore, it is necessary to measure a conductivity of the pure water just after being taken from the line, or in the line using a measuring electrode installed within the line (i.e., perform an in-line measurement).

In the United States Pharmacopeia (USP), a total organic carbon (TOC) measurement and a conductivity measurement are designated as testing standards for pure water and pharmaceutical waters. The TOC measurement is used as a guideline for measurements of organic carbon concentration, and the conductivity measurement is used as a guideline for measurements of inorganic ions concentration.

In the conductivity measurement, a three-stage philosophy is set out. In Stage 1, it is determined whether a conductivity of prepared pure water is not greater than a reference value of 1.3 μS/cm (at 25° C.), based on an in-line measurement which is performed while keeping the pure water from contacting ambient air or a laboratory measurement which is performed just after sampling the pure water. When the conductivity is not greater than the reference value, the test is completed. If the conductivity is greater than the reference value, the test will advance to Stage 2. In Stage 2, it is determined whether a conductivity of the sample under the condition of the sample is equilibrated with air is not greater than a reference value of 2.1 μS/cm (at 25° C.). When the conductivity is not greater than the reference value, the test is completed. If the conductivity is greater than the reference value, the test will advance to Stage 3. In Stage 3, saturated potassium chloride (KCl) is added to the water sample used in Stage 2 to adjust pH at a predetermined value, and it is determined whether a conductivity of the sample having the predetermined pH is not greater than a reference value, based on the laboratory measurement (see USP <645>).

Generally, an aqueous sample contains organic carbon (OC) formed as an organic compound bonded with oxygen, hydrogen, etc., and inorganic carbon (IC) as a constituent element of an inorganic compound. The amount of the organic compound is referred to as “total organic carbon (TOC) content”, and a sum of TOC and the amount of IC is referred to as “total carbon (TC) content”. An amount of carbon dioxide generated as a result of oxidation of carbon components in an aqueous sample is equivalent to a TC content of the aqueous sample.

For example, in a measurement of a TOC content or concentration, carbon components in a predetermined amount of aqueous sample are entirely oxidized and converted to carbon dioxide (CO₂) to measure a TC concentration. Then, a small amount of acid is added to a predetermined amount of the aqueous sample to acidify the aqueous sample. The acidified aqueous sample is subjected to an aeration or bubbling treatment using purified air to convert IC in the aqueous sample to CO₂ and transfer the CO₂ to the gaseous phase, and the CO₂-contained gas is led to a detector to measure an IC concentration. Subsequently, the IC concentration is subtracted from the TC concentration to obtain a TOC concentration.

Heretofore, the conductivity measurement and the TOC measurement have been performed using separate measurement apparatuses, respectively.

In a measurement for an aqueous sample, such as pure water, which has a relatively high capacity to absorb carbon dioxide gas, the conventional process of measuring a TOC content (or concentration) and a conductivity of an aqueous sample using separate measurement apparatuses is likely to cause a problem that carbon dioxide gas in ambient air is dissolved in the aqueous sample during the measurement to inadequately increase a conductivity of the aqueous sample beyond a reference value set forth by the USP. Moreover, the TOC measurement is generally a time-consuming operation. Thus, if it is tried to simultaneously measure TOC concentration and conductivity for a great number of aqueous samples, carbon dioxide gas in ambient air will be dissolved in the aqueous samples during the measurement.

### SUMMARY OF THE INVENTION

In view of the above circumstances, it is an object of the present invention to provide a water quality analyzer having a unitary structure capable of performing both a TOC measurement and a conductivity measurement while preventing carbon dioxide gas in ambient air from being dissolved in an aqueous sample.

In order to achieve this object, the present invention provides a water quality analyzer which comprises: a total organic carbon/conductivity measurement device provided with a total organic carbon measurement section including a mechanism for converting a carbon component in an aqueous sample to carbon dioxide and measuring an amount of the carbon dioxide, and a conductivity measurement section for measuring a conductivity of an aqueous sample; a gas aeration mechanism for passing a degassing gas devoid of carbon dioxide gas through an aqueous sample to perform a degassing treatment of expelling carbon dioxide gas in the aqueous sample; and a control section for controlling respective operations of total organic carbon measurement and conductivity measurement in the total organic carbon/conductivity measurement device. The control section is operable, just before measuring a conductivity of a specific aqueous sample during the conductivity measurement for the specific aqueous sample, to controllably instruct the gas aeration mechanism to pass the degassing gas through the specific aqueous sample so as to perform the degassing treatment.

In a specific embodiment of the present invention, the gas aeration mechanism is installed within the total organic carbon/conductivity measurement device, and designed to pass the degassing gas through an aqueous sample introduced in the total organic carbon/conductivity measurement device for the conductivity measurement, so as to perform the degassing treatment.

In the above specific embodiment, the total organic carbon/conductivity measurement device may be provided with a sampling syringe for receiving therein an aqueous sample, and designed to introduce an aqueous sample into the sampling syringe and selectively supply the aqueous sample to either one of the total organic carbon measurement section and the conductivity measurement section. In this case, the gas aeration mechanism may be connected to the sampling syringe in such a manner as to supply the degassing gas to the sampling syringe.

In another specific embodiment of the present invention, the gas aeration mechanism is located outside the total organic carbon/conductivity measurement device, and designed to pass the degassing gas through an aqueous sample just before being introduced in the total organic carbon/conductivity measurement device for the conductivity measurement, so as to perform the degassing treatment.

As above, the water quality analyzer of the present invention is provided with the gas aeration mechanism for passing the degassing gas through an aqueous sample just before the conductivity measurement to expel carbon dioxide (CO₂) gas dissolved in the aqueous sample therefrom. Thus, even if an aqueous sample contains carbon dioxide gas dissolved from ambient air due to storage for a long period of time after sampling, the carbon dioxide gas can be expelled to perform a desired conductivity measurement without adverse effects of the carbon dioxide gas.

The gas aeration mechanism may be installed within the total organic carbon/conductivity measurement device to completely eliminate the risk that an aqueous sample comes into contact with ambient air during a period from the degassing treatment to the conductivity measurement.

Further, in the total organic carbon/conductivity measurement device provided with the sampling syringe, the gas aeration mechanism may be connected to the sampling syringe to perform the degassing treatment. This makes it possible to perform both the degassing treatment and the introduction of an aqueous sample using the same sampling syringe so as to achieve structural simplification.

The gas aeration mechanism may be located outside the total organic carbon/conductivity measurement device. In this case, even if the total organic carbon/conductivity measurement device is not designed to internally perform the degassing treatment, the degassing treatment can be desirably performed.

### BRIEF DESCRIPTION OF THE DRAWINGS

FIG. 1 is an external view showing a total organic carbon/conductivity measurement device and an automatic sampler in a water quality analyzer according to one embodiment of the present invention.

FIG. 2 is a block diagram showing the configuration of the total organic carbon/conductivity measurement device in the water quality analyzer according to the embodiment.

FIG. 3 is an external view showing a water quality automatic sampler according to another embodiment of the present invention.

### DESCRIPTION OF THE EXEMPLARY EMBODIMENTS

The present invention will now be specifically described based on an embodiment thereof

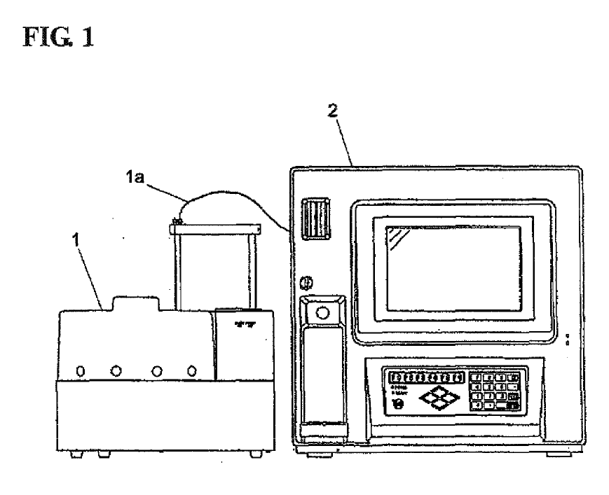

FIG. 1 is an external view showing a water quality analyzer according to one embodiment of the present invention, wherein a total organic carbon (TOC)/conductivity measurement device 2 (on the right side of the figure) having a conductivity measurement function in addition to a TOC measurement function, and an automatic sampler 1 (on the left side of the figure) is connected to each other through a sampling needle and a tube 1a. The total organic carbon/conductivity measurement device 2 is designed to internally subject an aqueous sample to a degassing treatment.

The automatic sampler 1 has a turntable capable of being loaded with seventy eight glass sample containers each having a capacity of 5 mL, and eight glass sample containers each having a capacity of 40 mL. A cap with a silicone rubber septum is attached to each of the sample containers.

The automatic sampler 1 and the total organic carbon/conductivity measurement device 2 is connected to each other through a flow passage defined in a PTFE (polytetrafluoroethylene) tube 1a having an inner diameter of 1 mm and an outer diameter of 2 mm.

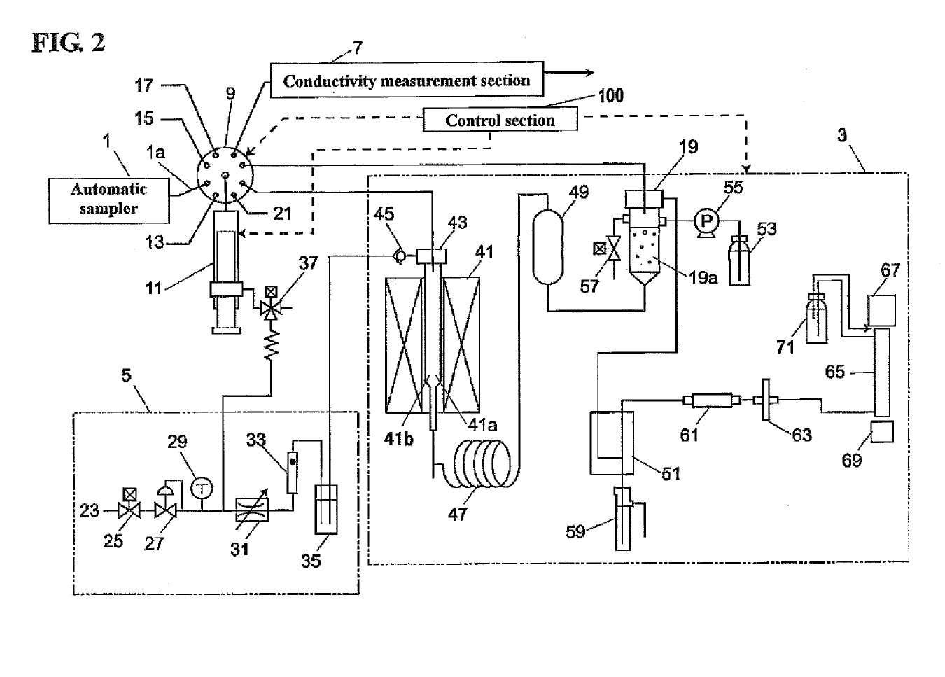

FIG. 2 is a block diagram showing the configuration of the total organic carbon/conductivity measurement device 2.

The total organic carbon/conductivity measurement device 2 comprises a TOC measurement section 3, a carrier gas supply section 5 for supplying a carrier gas into a total carbon (TC) combustion tube 41a installed in the TOC measurement section 3, a conductivity measurement section 7, and a multi-port valve 9 for switching each of the TOC measurement section 3, the carrier gas supply section 5 and the conductivity measurement section 7.

The multi-port valve 9 has a common port which is connected to a sampling syringe 11 for receiving therein a predetermined amount of aqueous sample, and the remaining ports which are connected, respectively, to the automatic sampler 1, a sample introduction section 13, a hydrochloric-acid supply section 15 for supplying hydrochloric acid for use in removing inorganic carbon (IC) components from an aqueous sample, a dilution-water supply section 17, an IC reactor 19, a conductivity measurement cell of the conductivity measurement section 7, the TC combustion tube 41a, and a drain line 21. The multi-port valve 9 is designed to supply an aqueous sample introduced from the automatic sampler to either one of the TC combustion tube 41a of the TOC measurement section 3, and the conductivity measurement section 7.

The conductivity measurement cell of the conductivity measurement section 7 is a flow type having a pair of platinum electrodes within a cylindrical-shaped silica chamber having an inner diameter of 2 mm.

The sampling syringe 11 has a cylinder capacity of 5 mL, and an aeration gas inlet is formed in a lower portion of a cylinder barrel thereof to receive a carrier gas therethrough. The aeration gas inlet is connected to the carrier gas supply section 5 through a solenoid valve 37. In this embodiment, the sampling syringe 11 serves as a part of a gas aeration mechanism.

The carrier gas supply section 5 is designed to supply highly-pure air as a carrier gas. Specifically, the carrier gas supply section 5 comprises a carrier gas inlet 23, a solenoid valve 25, a pressure regulator valve 27 for adjusting a pressure of the carrier gas, a pressure meter 29 for measuring the adjusted pressure, a mass flow controller 31 for adjusting a flow rate of the carrier gas, a flowmeter 33, and a humidifier 35, which are interposed or connected in/to a carrier gas flow passage in this order from an upstream side thereof. The carrier gas after being set at a predetermined flow rate and humidified is fed to the TC combustion tube 41a. Further, the carrier gas after being set at a predetermined flow rate and humidified is supplied as an aeration gas to the sampling syringe 11 through the solenoid valve 37.

The TC combustion tube 41a has a sample injection portion 43 at an upper end thereof, and a tubular-shaped portion whose lower end is filled with an oxidation catalyst 41b made of a metal oxide and/or noble metal to entirely convert carbon components in an aqueous sample to CO₂, and an electric heater 41 for heating the tubular-shaped portion. The sample injection portion 43 is connected to the carrier gas supply section 5 through a check valve 45 for preventing backflow of the carrier gas. The TC combustion tube 41a has a lower outlet which is connected to a carrier gas inlet of the IC reactor 19 through a cooling tube 47 and an anti-backflow trap 49.

In an operation of measuring an IC concentration (IC measurement), a pump 55 is activated to supply phosphoric acid as an IC reactive liquid 19a from a phosphoric acid reservoir 53 to the IC reactor 19. Further, an aqueous sample is directly injected into the IC reactor 19. Thus, IC in the injected aqueous sample is converted to CO₂, and the generated CO₂ is led to a electronic dehumidifier 51 by the carrier gas. After the reaction, a draining solenoid 57 is opened to discharge the IC reactive liquid 19a from the IC reactor 19.

The gas passing through the electronic dehumidifier 51 is led to a nondispersive infrared (NDIR) analysis-based cell 65 through a reserver 59 for pooling removed moisture from the gas, a halogen scrubber 61 for removing halogen components from the gas, and a membrane filter 63 for removing foreign substances from the gas. A light source 67 and a detector 69 are disposed, respectively, at opposite ends of the cell 65 in opposed relation to each other. A signal from the detector 69 is equivalent to an IC concentration of the aqueous sample. Then, the carbon dioxide gas is discharged from the cell and absorbed in a CO₂ absorber 71. A drain pot serving as the reserver 59 is connected to a bottom of the electronic dehumidifier 51.

Each of the TOC measurement section 3, the multi-port valve 9, the sampling syringe 11 and the solenoid valve 37 for opening and closing the carrier gas flow passage to the sampling syringe 11 is connected to a control section 100. The control section 100 is operable to control respective operations of TOC measurement and conductivity measurement. Specifically, the control section 100 is operable, just before measuring a conductivity of an aqueous sample during the conductivity measurement for the aqueous sample, to controllably instruct the multi-port valve 9, the sampling syringe 11 and the solenoid valve 37 to pass a degassing gas (i.e., carrier gas) through the aqueous sample so as to perform a degassing treatment. After the degassing treatment, the carrier gas supplied to the sampling syringe 11 is discharged from a drain port of the multi-port valve 9 connected to the drain line 21.

An operation of the water quality analyzer according to this embodiment will be described below. While the conductivity measurement and the TOC measurement in a water quality analysis of an aqueous sample may be performed in any order, the following description will be made based on one example where the conductivity measurement is initially performed, and then the TOC measurement is performed.

[1] Conductivity Measurement

2 mL of water sample (e.g., pharmaceutical pure water) is introduced from one of the sample containers in the automatic sampler 1 into the sampling syringe 11 through the sampling needle and the tube 1a, according to a suction force of the sampling syringe 11.

After the aqueous sample is received in the sampling syringe 11, a plunger of the sampling syringe 11 is moved downwardly to a position below the aeration gas inlet formed in the lower portion of the cylinder barrel, and the solenoid valve 37 is opened. Thus, highly-pure air serving as the carrier gas is introduced into the sampling syringe 11 at a flow rate of 100 mL/min to subject the aqueous sample in the sampling syringe 11 to the aeration (i.e., degassing) treatment. Then, the carrier gas is discharged from the drain port of the multi-port valve 9. The degassing treatment is performed, for example, for 1.5 minutes. Through the degassing treatment, carbon dioxide gas dissolved in the aqueous sample is expelled therefrom. The above operation of the degassing treatment is controlled by the control section 100.

After the degassing treatment, the ports of the multi-port valve 9 are switched to connect the sampling syringe 11 to the conductivity measurement section 7. Then, the plunger of the sampling syringe 11 is moved upwardly to inject the degassed aqueous sample into the conductivity measurement cell of the conductivity measurement section 7 so as to measure a conductivity of the aqueous sample.

[2] TOC Measurement

After the aqueous sample is re-sucked from the automatic sampler 1 into the sampling syringe 11, the ports of the multi-port valve 9 are switched to connect the sampling syringe 11 to the TC combustion tube 41a. Then, the plunger of the sampling syringe 11 is moved upwardly to supply the aqueous sample to the sample injection portion 43 of the TC combustion tube 41a. Simultaneously, highly-pure air serving as the carrier gas is fed from the carrier gas supply section 5 to the sample injection portion 43 through the check valve 45, and therefore the aqueous sample and the air is injected into the TC combustion tube 41a. In the TC combustion tube 41a, the tubular-shaped portion is heated up to 680° C. by the electric heater to oxidize and convert carbon components of the aqueous sample to carbon dioxide.

Gases (carbon dioxide gas and water vapor) generated in the TC combustion tube 41a are cooled by the cooling tube 47, and therefore only the carbon dioxide gas is led to the IC reactor 19 through the anti-backflow trap 49. The carbon dioxide gas passing through the IC reactive liquid 19a is led from an upper portion of the IC reactor 19 to the electronic dehumidifier 51 to remove moisture therefrom. After further passing through the halogen scrubber 61 and the membrane filter 63 to remove halogen components and foreign substances, the carbon dioxide gas is led to the cell 65. Then, in response to infrared light emitted from the light source 67 into the cell 56, a signal proportional to a concentration of carbon dioxide in the gas is obtained from the detector 69. This signal is equivalent to a TC concentration of the aqueous sample. Then, the carbon dioxide gas is discharged from the cell and absorbed in the CO₂ absorber 71.

Subsequently, after the aqueous sample is re-sucked from the automatic sampler 1 into the sampling syringe 11, the aqueous sample is injected into the IC reactor 19 according to a switching operation of the multi-port valve 9 and an upward movement of the sampling syringe 11. In the IC reactor 19, the carrier gas is introduced from a bottom of the IC reactor 19 to allow the IC reactive liquid 19a to be kept in a bubbling state. The aqueous sample injected from the upper portion of the IC reactor 19 in the bubbling state comes into contact with the IC reactive liquid 19a, i.e., phosphoric acid solution, to generate carbon dioxide through acidification. The gas containing the generated carbon dioxide is led to the electronic dehumidifier 51 to remove moisture therefrom. After further passing through the halogen scrubber 61 and the membrane filter 63 to remove halogen components and foreign substances, the carbon dioxide gas is led to the cell 65. Then, in response to infrared light emitted from the light source 67 into the cell 56, a signal proportional to a concentration of carbon dioxide in the gas is obtained from the detector 69. This signal is equivalent to an IC concentration of the aqueous sample.

Then, a TOC concentration of the aqueous sample can be obtained by subtracting the IC concentration from the TC concentration measured in the above manner.

The TOC/conductivity measurement device 2 in this embodiment is equipped with a mechanism for supplying an aeration (i.e., degassing) gas to the sampling syringe 11 and a mechanism for supplying acid to the sampling syringe 11. Thus, a TOC concentration of an aqueous sample can be measured directly (i.e., in a single operation). Specifically, after an aqueous solution is introduced into the sampling syringe 11, the ports of the multi-port valve 9 are switched to supply hydrochloric acid from the hydrochloric-acid supply section 15 to the sampling syringe 11, and the sampling syringe 11 is moved to suck the hydrochloric acid therein. Then, the ports of the multi-port valve 9 are switched to connect the sampling syringe 11 to the drain port, and the sampling syringe 11 is further moved downwardly to the position below the aeration gas inlet formed in the lower portion of the cylinder barrel. The solenoid valve 37 is then opened to introduce highly-pure air serving as the carrier gas into the sampling syringe 11 so as to subject the aqueous sample in the sampling syringe 11 to the aeration treatment. Then, the carrier gas is discharged from the drain port of the multi-port valve 9. In this discharge operation, IC dissolved in the aqueous sample, together with the carrier gas, is discharged as a carbon dioxide gas. Subsequently, the aqueous sample is led to the TC combustion tube 41a, and then carbon components in the aqueous sample are measured to determine a TOC concentration thereof.

A water quality analyzer according to another embodiment of the present invention will be described below.

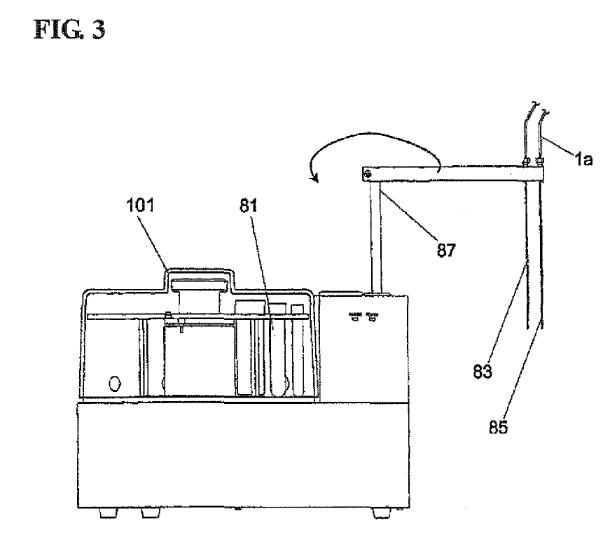

FIG. 3 is a schematic diagram showing the water quality automatic sampler according to this embodiment, wherein an aeration (i.e., degassing) treatment for an aqueous sample is performed in an automatic sampler. While a total organic carbon/conductivity measurement device in this embodiment may have the same configuration as that illustrated in FIG. 2, the carrier gas flow passage connecting between the carrier gas supply section 5 and the sampling syringe 11 can be omitted.

In an automatic sampler 101 illustrated in FIG. 3, the reference numeral 81 indicates a sample container which contains an aqueous sample to be measured. The reference numeral 85 indicates a sampling needle which is designed such that a lower end thereof is moved to a target position by a needle drive unit 87 so as to suck the aqueous sample from the sample container 81. The sampling needle 85 has an upper end which is connected to a flow passage 1a in fluid communication with the total organic carbon/conductivity measurement device so as to allow the sucked aqueous sample to be introduced into the measurement device.

In this embodiment, the automatic sampler 101 is provided with an aeration needle 83. The aeration needle 83 is designed to inject an aeration (i.e., degassing) gas from a lower end thereof into the sample container 81 before the aqueous sample is sucked by the sampling needle 85, so as to subject the aqueous sample to an aeration (i.e., degassing) treatment. The aeration needle 83 has an upper end connected, for example, to the carrier gas supply section 5 (see FIG. 2) for supplying a carrier gas.

An operation of the water quality analyzer according to this embodiment will be described below.

The aeration needle 83 is inserted into a target sample container 81 to inject a carrier gas, such as highly-pure air, thereinto. The aeration gas comes in contact with the aqueous sample to expel carbon dioxide gas from the aqueous sample through the gas-liquid contact therebetween, and the carbon dioxide gas is discharged outside from an upper portion the sample container 81.

After completion of the degassing treatment using the aeration gas, the aeration needle 83 is pulled up out of the sample container 81, and then the sampling needle 85 is promptly inserted into the sample container 81 to introduce the degassed aqueous sample into the conductivity measurement section 7 (see FIG. 2) through the flow passage 1a so as to perform the conductivity measurement.

### INDUSTRIAL APPLICABILITY

The water quality analyzer of the present invention is useful for managing target water based on conductivity.

What is claimed is:

1. A water quality analyzer comprising:
a total organic carbon/conductivity measurement device provided with a total organic carbon measurement section including a mechanism for converting a carbon component in an aqueous sample to carbon dioxide and measuring an amount of the carbon dioxide, and a conductivity measurement section for measuring a conductivity of an aqueous sample;
a gas aeration mechanism for passing a degassing gas devoid of carbon dioxide gas through an aqueous sample to perform a degassing treatment of expelling carbon dioxide gas in the aqueous sample; and
a control section for controlling respective operations of total organic carbon measurement and conductivity measurement in said total organic carbon/conductivity measurement device, said control section being operable, just before measuring a conductivity of a specific aqueous sample during the conductivity measurement for said specific aqueous sample, to controllably instruct said gas aeration mechanism to pass the degassing gas through said specific aqueous sample so as to perform said degassing treatment.

2. The water quality analyzer as defined in claim 1, wherein said gas aeration mechanism is installed within said total organic carbon/conductivity measurement device, and designed to pass the degassing gas through an aqueous sample introduced in said total organic carbon/conductivity measurement device for the conductivity measurement, so as to perform said degassing treatment.

3. The water quality analyzer as defined in claim 2, wherein:
said total organic carbon/conductivity measurement device is provided with a sampling syringe for receiving therein an aqueous sample, and designed to introduce an aqueous sample into said sampling syringe and selectively supply said aqueous sample to either one of said total organic carbon measurement section and said conductivity measurement section; and
said gas aeration mechanism is connected to said sampling syringe in such a manner as to supply the degassing gas to said sampling syringe.

4. The water quality analyzer as defined in claim 1, wherein said gas aeration mechanism is located outside said total organic carbon/conductivity measurement device, and designed to pass the degassing gas through an aqueous sample just before being introduced in said total organic carbon/conductivity measurement device for the conductivity measurement, so as to perform said degassing treatment.
